(19) United States
(12) Patent Application Publication
(10) Pub. No.: US 2003/0007892 A1
(43) Pub. Date: Jan. 9, 2003

(54) UA cup

(76) Inventor: Jack V. Smith, Arden, NC (US)

Correspondence Address: JACK V. SMITH, P.O. BOX 156, Arden, NC, 28704, US

(21) Appl. No.: 09/901,160

(22) Filed: Jul. 9, 2001

(51) Int. Cl. G01N 33/48

(52) U.S. Cl. 422/58; 422/61; 436/164; 436/166; 436/165

(57) ABSTRACT

The present invention is a device for assaying and collection of biological and other specimens and is especially designed for the collection and determination of the presence of chemical constituents in clinical chemistry, drugs of abuse testing, infectious disease testing, and other fields of analysis of fluids.

### BACKGROUND OF THE INVENTION

1. Field of the Invention

The present invention is a method that elates to assaying and collecting biological and other specimens and is especially designed for the collection and determination of the presence of chemical constituents in drugs of abuse, urinalysis, infectious disease, clinical chemistry and other areas of analysis The collection devices of the prior art for urine for example were not designed to be used for analysis. These devices were strictly designed to collect urine on the ward of a hospital and then sent to the laboratory for testing. Or, the nurse would collect a urine and take it back to the nurse's station and test the urine commonly with a urine dipstick. There are several drawbacks to this. First the nurse will have to have an open urine container at the nurses station. This presents a biological hazard that the nurse, doctors, patients, and passerby's would be exposed to. The chances are spillage of the urine specimen or any liquid for that matter is high when ever you have an open container present. With this specimen now present at the nurses station after collection the pressure to test and dispose of the specimen is increased for workload, safety and storage area (clutter) reasons alone. The next step for the nurse would be to take the urine specimen and dip a dry chemistry test strip into the urine and analyze for urine analytes of interest (constituents). The constituents that are commonly analyzed in urine specimens are glucose, pH, specific gravity, bilirubin, urobilinogen, nitrite, protein, red blood cells (hemoglobin), ketones, and white blood cells (human leukocyte esterase). Once the test strip has been removed from the specimen it needs to be compared to a color chart to determine the concentration of the urinary constituents. The nurse will then wait the pre-required time to read each and every color pad as designated by the package insert for the test strip by the manufacturer. After analysis the nurse does not want to lay this strip on the counter for contamination reasons. The nurse may possibly use a paper towel to lay the strip on. Once the results are recorded the nurse will then properly dispose of the test strip and urine specimen and container. Resulting in an inordinate amount of risk, time, and labor.

2. Description of the Related Art

The present is device that is designed to collect and assay the presence of urinary constituents (analytes of interest) in a biological urine specimen. This specimen could come from humans, animals or other sources submitted for analysis of the analyte of interest. That is to say for example that the present device (invention) is designed to be used for the collection and detection of glucose in the urine specimen or the device is used to collect urine and detect virulent disease causing viruses such as HIV, proteins, viruses, drugs of abuse, drug metabolites, clinical analytes of interest, and therapeutic drugs.

There is no prior that produces the unexpected results of the present device and the answers to a solution to that was never before even recognized that the present art provides. The prior art teaches away from the present art in that it goes in a completely different direction. That is to say that the collection devices of the past for urine were not designed to be used for analysis but strictly collection. For example these devices were strictly designed to collect urine on the ward of a hospital and then be sent to the laboratory for testing. The collection device was designed to collect urine and test however these devices are cumbersome, expensive, and not designed for the specific purpose of testing biological constituents. These some devices are designed to perform analysis of certain constituents but in a cumbersome and messy manner and these devices were not designed to collect urine for any period time and have numerous drawbacks and limitations when related to the advance that the present device brings to the art.

A thorough search of patents, publications, and research revealed no relative art (i.e., prior art) showing any direct correlation to this novel invention. The search included the USPTO (United States Patent Office) data base with no patents issued for a device designed specifically for biological specimen or other fluid collection and testing that is unique this device. However, the following art will be mentioned to further illustrate the novelty of the present art and the obvious advancement to the current art. The following patents, without exception do not mention the use of a cup for collection and analysis of biological specimens for detecting specific analytes of interest.

It is known in the art that the urine matrix is very complex and consists of many urinary constituents which create strong buffering and interference problems (e.g. cannibal-like enzymes such as protease) that have to be overcome to provide a method that can be used for the general population with precision and accuracy. Simply because a technique can accommodate a liquid sample does not imply that it can be successfully used with any liquid test matrix. Such successful adaptation of test techniques to accurately deal with specific sample matrices aren't often “obvious” to any scientist. The same can be said of certain types of techniques used to analyze urine. For instance, the art is replete with examples of devices that provide dry chemistry dipsticks for dipping into a urine container and reading the result. However these dipsticks devices have crossover contamination problems from reaction pad to reaction pad because the dipstick is covered with urine and the urine from back and forth from reaction pad to reaction pad. However, the present art will demonstrate in detail the techniques developed that will overcome these type of interferences and issues with the prior art.

The number of collections of biological specimens is very large in the United States and worldwide. The numbers are in the hundreds of thousands of specimens per day collected in urine containers for drugs of abuse screening, adulteration testing, urinalysis, infectious disease testing, clinical chemistry and other testing. Since very large numbers collected are involved it is very important to the art for a device designed to answer the problems of the current art that will be effective, safe, simple, and cost effective. No current device in the art solves these problems until this invention.

Specimens collected for drugs of abuse testing sometimes require that the specimen integrity and chain of custody be validated. The adulteration of samples submitted for drug testing is unacceptable. The assay run on any specimen submitted for any analysis is only as good as the specimen collected.

Also with the onset of HIV (human immunodeficiency virus), STD's (sexually transmitted diseases), hepatitis and other infectious diseases the health risk associated with the handling of body fluids has increased exponentially over the last few years. Therefore, if a device is invented that can provide added safety it is very likely that it will save lives.

The multiple steps of specimen collection as required with the prior art are hazardous with regards to infectious diseases. First the sample is collected in a container then the specimen is transferred to another container for testing in a device, test tube, or instrument. In the case of drugs of abuse testing the sample has to be split to another container before it is tested so that the original container is not contaminated with the test device (in case of cross contamination from the test device). These multiple steps procedures of potentially infectious material have required the manual use of test tubes, pipettes, syringes, or other devices used in the transfer of specimens from collection device to the final container use for analysis. Then of course after the assay is completed the assay container and or the specimen has to be discarded.

Another issue with the prior art is the possible misidentification or mislabeling of the specimen collected anytime the specimen has to be removed from the original container. This could in an erroneous result for the original specimen. Imagine a urine submitted for an HIV test and it was mix up with another specimen because of mislabeling and as erroneous result was reported. The implications are grave.

Different attempts at providing an effective collection device have been attempted but all have failed for multiple reasons. U.S. Pat. No. 5,403,551 to Galloway, describes a cup for collecting and analyzing a specimen but this device has multiple drawbacks. The device requires that the user to invert the container prior to analysis. When the container is inverted it leaks quite profusely. Which does not answer the contamination problem. The device is assay part of the device is attached to the collection cup and is not part of the cup and requires a number of chambers, channels, and other means that add to the cost and complexity of the device. In addition, the Galloway device requires the use of a plenum (a space in which a gas, usually air, is contained at a pressure greater than atmospheric pressure. In, addition, U.S. Pat. Nos. 5,096,813 and 4,769,215 to Ehrenkranz provides drug testing urine collector type devices that includes perhaps the most complex devices ever designed for urine collection. The complexity of the devices alone would raise the cost of the devices to a level that it would infeasible to market and sell the devices. The devices have almost as many problems as the Galloway device. They actually has adulteration detection reagents in the reservoir. This is a major problem with regard sample contamination. The complexity of manufacturing and the contamination issues from the adulteration detection reagents to name a few are major drawbacks to these devices. U.S. Pat. No. 5,096,813 to Krumhar is a device designed specifically to for storage and the detection of oxygen and has no relative bearing on the present invention. It is however, a device used for storage and by no means can be compared to the present device which can analyze a specimen at the point of collection, without tilting the cup, or pouring into another device, etc.

While the prior art provides certain devices for the collection of fluids or other types of samples the prior art however suffers from a certain number of drawbacks.

The inflation, insertion, and closure of the prior art devices all require multiple steps and are not simple efficient method to collect and analyzed urine without the risk or contamination, spillage, or other problems. All of the prior art requires tedious and complex methods for use. For instance, the one prior requires that certain the cup be tilted prior to analysis increasing the risk of leakage and contamination as the specimen leaks out of the container. Another device requires the use of a plunger (syringe) for use and yet another requires the use of tilting and a plenum. These are just some, not all, of the limitations of the prior art.

The present device is designed for the analysis of biological specimens on site. That is to say the device can be used for the collection and analysis of the specimen within the container without removal of the specimen and without have to adjust the lid of the container, use a plunger, a plenum, or other multiple steps as required by the prior art. The specimen can be analyzed immediately at the point of collection or sent to the lab and tested the next day. The device can be used for long storage of a specimen before testing and/or for immediate analysis. This removes the risk of contamination, mislabeling, chain of custody, and cross over contamination.

### SUMMARY OF THE INVENTION

The present invention is designed to advance the art of urine collection and on site (at the point of collection) analysis past the prior arts drawbacks and provide a collection and analyzing cup that is simple to use, requires minimal instruction, has the minimum number of parts, and is cost effective. Another object of the present invention is to provide a method that allows for an easily automated process and readable.

Correspondingly, another advantage of the present art is to provide a collection and analyzing device that will allow the user to collect the specimen in the cup, place the lid on the cup to prevent any biohazard accidents or contamination, analyze the specimen without having to further manipulate the cup like tilting, using a plunger, a plenum, etc. This is truly a one step process which is not currently known in the art.

In addition the present art provide a unique activation device which is designed to prevent accidental activation of the integrated analyzing component of the cup.

It has been found that the foregoing objects of the present art are accomplished in accordance with this invention by providing a collection and analyzing cup that is designed to collect the specimen and immediately have the lid secured onto the top of the cup. Then the user can analyze the specimen when ready. The cup is designed for long term storage if necessary before and after analysis.

The present invention provides a method of specimen collection and analysis as defined above, and the method being characterized by the following steps:

a) collecting the specimen in the cup;

b) placing the lid on the cup and closing;

c) activating the analyzing component of the cup by depressing the activation device;

d) and recording the results of the analysis without the use of a plunger or the requirement of tilting the specimen.

Other aspects and advantages of the present invention appear more clearly from reading the following detailed description of the preferred embodiment of the invention, given by way of example and made with reference to the accompanying drawings. Such as the determination of exactly how the device works. A thorough search of the literature reveals no relative art resembling this technology; therefore, this invention is clearly a novel in creation, and is not obvious to anyone skilled in the art, in fact the prior art devices teaches away from the present art in that the prior art requires that the cup be tilted in order for the device to be used (this is not a requirement of the present device in fact the present device is a teaching of simplicity with no manipulation of the cup as a requirement) and the prior art devices teach away from the present device in that some prior art require that the lid be off the container to activate, and the prior art teaches away from the present device in that the prior teaches the use and requirement of a plenum which is a pressurized space, etc. The present device teaches the use of a chamber that does not require pressure and a stable pressure to a vacuum would actually be preferred. There are certain aspects of the present art that can be found in the prior art (e.g. the use of a cup) but no prior has advanced the art of specimen collection and analysis as much as the present art. This art solves an unrecognized problem that was never before even recognized. Specifically this allows for the user the unexpected results of using a device that is simple, efficient and cost effective that only utilizes a cup and activation device without the use of plungers, plenums, tilting, etc., for a much more effective and safe method of collection and analysis of a specimen.

The collection and assaying device, in accordance with the present device, for both collecting and analyzing specimens, includes a container (cup) having an opening for collection of the specimen and a chamber for storing the collected specimen. A cap provides a means for sealing the container opening and an assay means which is not attached but integrated into the container providing for chemically analyzing the specimen.

The specimen can be a biological sample (urine, etc.) or other type of fluid.

It important that the means are provided for introducing a portion of the collected specimen within the chamber into the assay means when the cap on the container. However, this device and testing means does not require that the cap be in place. By placing the cap into position there is no requirement for removing the sample from the assaying device in order to conduct chemical analysis.

Therefore, the apparatus of the present device (invention) totally removes the need to transfer the collected sample from the device in order to conduct a chemical analysis as is the case with prior art devices. As mentioned this has a significant importance with regard to safety, biohazard, time and savings.

Additionally, one embodiment of the present device is particularly suitable for Infectious disease, Drugs of Abuse, Urinalysis Testing of biological fluids which includes a means for preventing premature or inadvertent entry of the specimen into the assay means (because of the special design of the activation means). And, since the fluid specimen never leaves the device, if a positive test for HIV (infectious disease), or drug, etc., is indicated, the entire device may be removed or shipped to the laboratory or other facility for further or confirmation testing.

Additionally, the present device, the assay means may include chromatograph, thin layer chromatography and dry chemistry hybrid, dry chemistry test pads attached to lateral flow device or other material assay means integrated in the container for enabling direct visual observation of the assay results. Therefore, no additional steps are necessary for effecting an analysis of a biological specimen.

As mentioned above, the assaying means of the device in accordance with the present invention includes a means for preventing biological fluid from entering the assay means during the collection of the body as is the case with all prior art (as discussed with the plenum and the tilting as required by one particular art (note: this could happen with the prior art during collection the cup could be tilted while the specimen is entering the cup and the specimen goes directly into the plenum). Therefore with the present invention it is very important to prevent premature or inadvertent activation of the assaying means and further the possibility of attempted adulteration the assaying means during collection which, is virtually eliminated by the present invention is a major problem with all of the prior art.

A wicking means may be provided but is not required for enabling the biological fluid to enter the assaying means or aid the assaying means in the movement of fluid from one end of the assaying means to the other.

The assaying means is integrated into the container side wall (not bottom or top), and the activation means include a spring loaded shaft that perforates the opposite side wall of the chamber allowing the entry of biological fluid once the shaft is activated and return to its original position after perforating the inside wall of the chamber allowing the biological fluid to enter the shaft chamber and coming into contact with assaying means.

The assaying means may contain a plurality of separated thin layer chromatograph strips with each strip comprising means for chemically analyzing the biological fluid for a different analyte, or chromatograph, or thin layer chromatography and dry chemistry hybrid, or dry chemistry test pads attached to lateral flow device or other material assay means.

And the assaying means may include a wick for evenly distributing the biological fluid to each of the assay means if more than one is present. The wick can be at both ends of the assaying means or at just one end of the assay means, or not present at all.

### BRIEF DESCRIPTION OF THE DRAWINGS

Other objects, features and advantages of the invention will become obvious from the following detailed description of the invention when taken in conjunction with the accompanying drawings, in which:

FIG. 1 is a plan view of one embodiment of the UA Cup made in accordance with this invention generally showing the container, activation device, and assaying means;

FIG. 2 is a cross section view of FIG. 1 prior to placing the lid onto the container generally looking down into the container from the top;

FIG. 3 is a plan view of the UA Cups lid which can be placed and screwed onto the top of the container;

FIG. 5 is a plan view of another embodiment of the UA Cup made in accordance with this invention generally showing the container, activation device, and assaying means.

### DETAILED DESCRIPTION OF THE PREFERRED EMBODIMENTS

The present invention will now be described more fully with reference to the accompanying drawings, in which the preferred embodiments of the present art invention are shown. It is understood from the embodiments that a person skilled in the art may make variations and modifications without departing from the spirit and scope of the invention. Such as changing the size or shape of a UA Cup, the optional addition of a wick, or the addition of a magnifying side wall to allow for easier reading and automation of the assaying mean results.

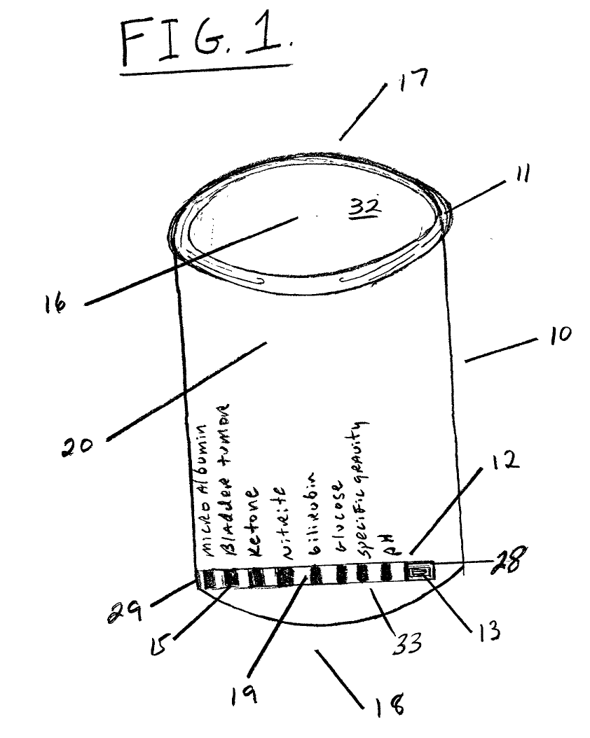

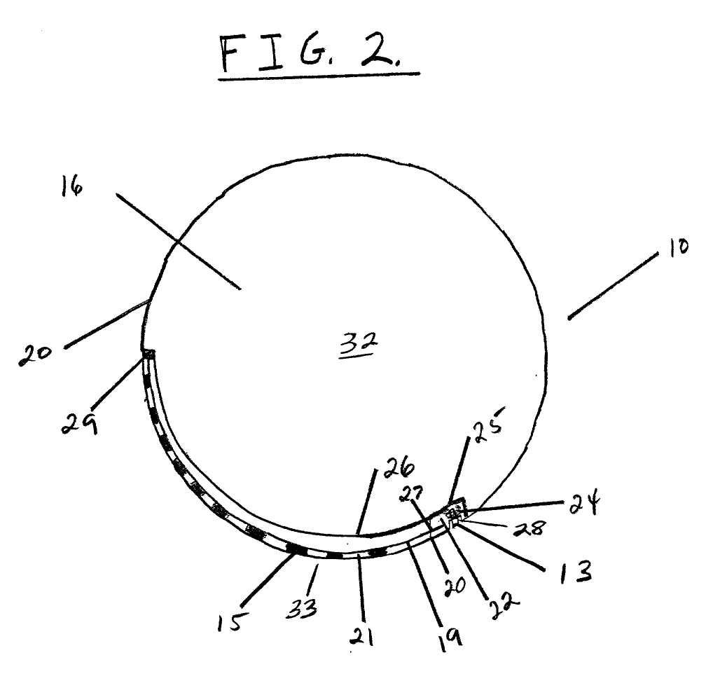

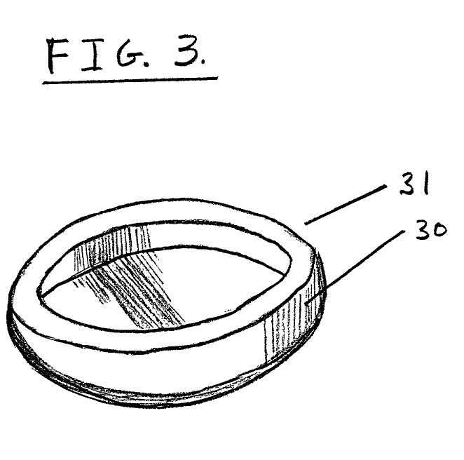

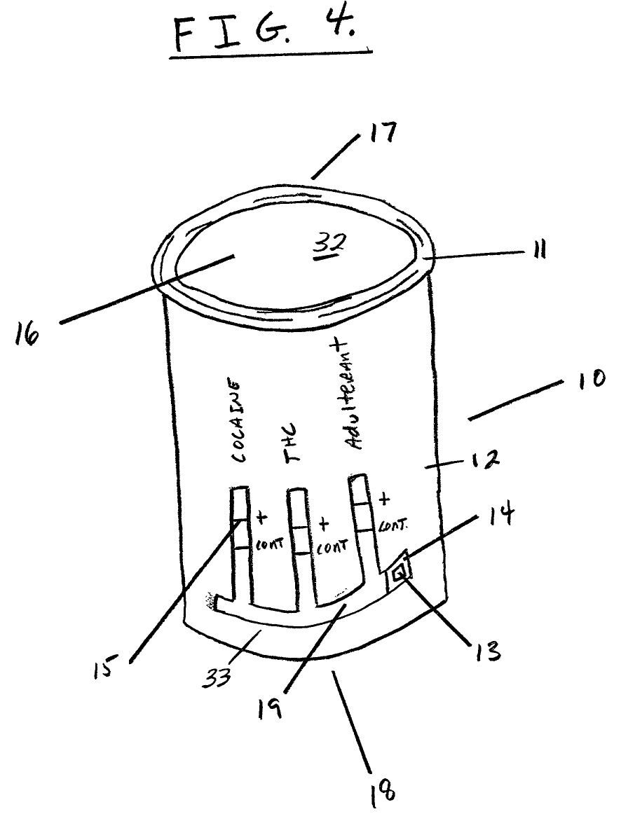

Referring now to the drawings and in particular FIG. 1 and 2, there is shown an collection and assaying device 10, in accordance with the present invention. The device generally includes a container 12 having an opening 16 which provides a means for collecting the biological fluid and a chamber 32 which provides a means for storing the collected fluid.

The container 12 and assaying means 33 may be formed, or molded, from any suitable material such as plastic, polymers, etc., and may include screw threads 11 at the top of the container 17 formed into the top 17 of the side walls 20 of the container opening 16 and are sized to for accepting the cap 31. The cap 31 when screwed onto the threads 11 provides a means for sealing the container 12 opening 16. For typical biological collection to include urinalysis (UA), drug screening, clinical chemistry, etc., the typical container 12 capacity of about 100 to 150 mLs to accommodate split specimen requirement and additional testing.

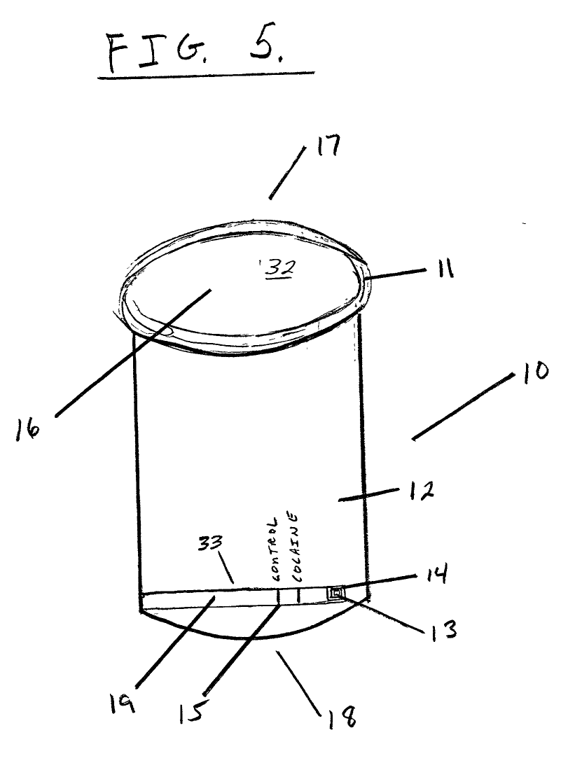

The assaying means 33 as shown in detail in FIG. 2 towards the bottom 18 of the container includes a spring loaded 24 shaft 13 which is recessed into the wall 28 of the container 12 to prevent accidental activation of the assaying means 33. The shaft 13 may be blunt or pointed so that it can perforate the opposite inner wall 25 of the chamber 32 and allow the fluid to enter the shaft chamber 22. The opposite inner wall may be thin to allow easier penetration of the shaft 13 through the inner wall 25 of the chamber 32. Once the shaft 14 has been depressed (activated) the inner wall 25 has becomes perforated and fluid enters the shaft chamber it will come into contact with the assaying means lateral flow material 19 (chromatographic, latex antibody strips, thin layer chromatography, lateral flow material, etc.) and the fluid will start to migrate to the opposite ends of the lateral flow means 19. During this time the fluid will come into contact with assaying means analyte detecting means 15 (such as dry chemistry test pads for glucose, nitrite, HIV, drugs of abuse, adulterants, etc., or lateral flow strips for drugs of abuse as shown in FIGS. 4 and 5) and a reaction will take place and a color or other detectable means will be measurable.

It should be acknowledge that any number of concurrent analyte specific tests may be performed with the device 10 of the present invention. While three assay analyte detecting means 15 strips as shown in FIG. 4 for three strips the device 10 only has to be one assay analyte detecting mean 15 as shown in FIG. 5. The device 10 may have a wick 29 as shown in FIG. 2. to aid in uptake of fluid from the shaft chamber 22 to the lateral flow material 19 dependent upon the number of assays performed on the device or the number of later antibody strips 15 as shown in FIG. 4 present if used, etc. The chamber 21 in which the assaying means 33 is located. This chamber is sealed as illustrated to prevent escape of any fluid from within the assaying chamber 21. Note, the only way for fluid to enter from the shaft chamber 22 to the assaying chamber 21 is via the lateral flow means 19. Also of note the lateral flow material means 19 can be backed (supported) with a plastic backing or solid plastic or other material means that may extend to the inner wall 26 of the assay means 33.

This detailed description as provided allows for a marked advance in the art of inflatable packaging. The steps are as follows:

a) collecting the specimen in the cup;

b) placing the lid on the cup and closing;

c) activating the analyzing component of the cup by depressing the activation device;

d) and recording the results of the analysis without the use of a plunger, plenum, or the requirement of tilting the specimen.

The simplicity and novelty of the invention is unmatched in the art. This device could be easily automated and include a magnifying plastic lens that would increase visibility of the assay means results. An automation example would be to have an instrument that depresses the activation device (shaft) then reads the bottom of the container automatically and download the result to a computer. This invention is going to save the clinical diagnostic, drug of abuse testing, and other industries millions of dollars in analysis time, safety prevention and accident control, time (labor), and cost through the novel simplicity of the present invention.

To further explain the

The invention has been described in detail with particular reference to a preferred embodiment and the operation thereof and it is understood that variations, modifications, and substitution of equivalent means can be effected and still remain within the spirit and scope of the invention. And all such modifications and variations are to be included within the scope of the invention as defined in the appended claims.

That which is claimed is:

1. An assaying device for collecting a fluid specimen, analyzing a portion of the sample said device comprising:
a) container means, having an opening, for collecting a specimen, and a chamber, for storing said specimen,;
b) cap means for sealing the container means opening;
c) assay means, integrated into the said container means, for chemically analyzing said specimen, said assay means being positioned in the outside wall of the container means for enabling direct visual observation thereof; and
d) means, for activating the said assay means by perforating the container means from the inside wall of the said container by a shaft means, allowing said specimen to enter said shaft chamber means, said last means comprising a lateral flow means connecting the said shaft chamber filled with said specimen and the assay means for providing fluid communication there between.

2. The assaying device according to claim 1 wherein said assay means comprises lateral flow means the allows fluid contact between the said shaft chamber means and the assay means.

3. The assaying device according to claim 1 wherein said assay means is integrated into the outside wall of the assay device.

4. A device for collecting and analyzing a fluid specimen, assaying a portion of the fluid specimen comprising;
a) containing means for collecting the said specimen;
b) placing said specimen into said containing means;
c) placing cap means for sealing onto the said container means;
d) depressing the activation means which contains a shaft means that perforates the inner wall of the said containing means and recording the results from the assaying means.
